(19) United States
(12) Patent Application Publication
(10) Pub. No.: US 2026/0249048 A1
(43) Pub. Date: Aug. 27, 2026

(54) MAGNETICALLY STEERABLE DEVICE FOR USE INSIDE OF A MAMMALIAN BODY

(71) Applicant: ETH ZÜRICH, Zurich (CH)

(72) Inventors: Roland DREYFUS, Zürich (CH); Sean Liam LYTTLE, Uster (CH); Christophe CHAUTEMS, Zürich (CH); Bradley NELSON, Zumikon (CH)

(73) Assignee: ETH ZÜRICH, Zurich (CH)

(21) Appl. No.: 18/874,716

(22) PCT Filed: Jun. 28, 2023
(86) PCT No.: PCT/EP2023/067573

§ 371 Date: Dec. 13, 2024

(30) Foreign Application Priority Data
Jul. 1, 2022 (EP) 22182617.5

(51) Int. Cl. A61M 25/01; A61B 1/005; A61B 1/01; A61M 25/00

(52) U.S. Cl. CPC A61M 25/0127; A61B 1/0051; A61B 1/01; A61M 25/0054; A61M 2025/006

(57) ABSTRACT

A steerable device for use inside of a mammalian body, including: a flexible elongated element and a magnetic element placed in a distal end portion of the elongated element to allow steering by way of an external magnetic field. The steerable device includes a structured section arranged proximal to the magnetic element and having a plurality of elongated protrusions formed on an outer surface of the elongated element. The elongated protrusions have each a protrusion longitudinal axis, each protrusion longitudinal axis forming an angle with a device longitudinal axis, wherein each angle is less than 90°, such that rotation of the elongated element in the clockwise direction around the device longitudinal axis effects a movement of advancement of the elongated element relative to the soft tissue. In a further aspect, the structured section is in the form of an outer thread structure.

The invention relates to a steerable device for use inside of a mammalian body. The device may be a needle, guidewire, catheter, endoscope, or any other flexible device designed to be inserted into a mammalian body for diagnostic or interventional purposes.

Many medical procedures require a medical instrument to be navigated to a specific location inside the body. Examples include deep brain stimulation, organ biopsies, targeted drug delivery, tumor removal, and many others. The insertion and navigation of such devices is achieved in a variety of ways depending on the application.

In the case of procedures inside organs, such as the brain or liver, a semi-rigid needle or guidewire is pushed from an incision point through the soft tissue of the organ to the target site. There is often little or no ability to steer or direct the needle so the path from incision point to target site is essentially a straight line. There are a very limited number of target sites that can be reached (it is very difficult for instance to reach target sites at large angles from the point of insertion). Because the devices are relatively rigid, navigating to different target sites inside an organ requires unique paths from the incision point to the target site. This can be very damaging to the tissue between the insertion point and the target sites. Friction between the soft tissue and the device's outer surface can also be a problem and buckling can often occur if the needle or guidewire is sufficiently flexible, further damaging the tissue and limiting the efficacy of the procedure.

In the case of procedures inside body cavities, such as the abdomen or inside the bladder, a flexible device is steered, usually by means of puller wires or rotating a pre-curved distal tip, while being pushed proximally. There is an inherent tradeoff between the device's ability to reach target sites and its ability to be advanced without buckling-if the device is sufficiently flexible to be steered to target sites at large angles from the insertion point, it is also more prone to buckling or forming loops when being pushed.

In the case of procedures in the vasculature, a catheter or guidewire is pushed proximally into the vessel and steered, again by means of puller wires or pre-curved tips, to the target site. Again, there is a tradeoff between the stiffness necessary to push without buckling and the flexibility needed to reach a target site. Because the device is advanced by pushing proximally, prolapse of the device often limits its ability to reach target sites deep in the vasculature and can be damaging to the vessel walls.

Some proposed devices have a magnetic device tip that allows a device to be more flexible and its trajectory to be more precisely controlled, but these devices still rely on pushing to make forward progress, and as such have all the difficulties listed above. Furthermore, the rigidity of the device necessary to make pushing possible at all greatly limits its ability to be steered magnetically.

U.S. Pat. No. 7,815,580 B2 discloses a magnetically navigated guidewire designed for enabling passage through occlusions in the vasculature. The guidewire consists of an elongated wire with a distal tip. Proximal to the tip, a magnetically responsive element is placed, which is responsive to an external magnetic field for steering the distal tip of the guidewire. An external helical thread is formed on the surface of the distal tip and is used to screw through a blockage in the occluded vessel. The guidewire is pushed through the vasculature normally until it encounters an occlusion. Then the guidewire is rotated such that the helical thread on the distal tip penetrates the occlusion. The occlusion can be removed along with the guidewire when it is pulled out of the vasculature or a stent can be placed inside of the occlusion. The helical thread extends only over a very small length at the distal tip and is solely designed for boring the tip into a blockage in the blood vessel. The guidewire's purpose is to install a stent to alleviate the occlusion of the vessel that is caused by the blockage. Therefore, the tip of the guidewire only needs to reach the blockage and pass through it with the help of the helical thread. The guidewire still relies on a push-to-advance mechanism for moving the tip of the guidewire through the vasculature up to the occlusion, and retracting the guidewire will require a pulling at the proximal end of the guidewire. The helical thread does not extend beyond the outer surface of the main guidewire body, so that it does not scrape or otherwise damage the tissue of the vessel wall. Further, the helical thread is solid to allow screwing in the blockage and not flexible as in the present invention.

U.S. 20100069718A1 discloses a different catheter for insertion into body cavities of a human. A distal section of the catheter is covered by a helical thread structure, allowing the catheter to be advanced by rotating the catheter, by way of a so-called rotate-to-advance mechanism.

U.S. 2005272976A1 discloses an endoscope insertion aiding device having a flexible tube and a distal-end member with the outer diameter equal to or more than the outer diameter of the tube at the distal end of the tube. The tube has a spiral structure on the outer circumferential surface thereof.

WO2021126905A1 and EP3643353A1 disclose a miniature device configured to be maneuvered within a patient under manipulation by an external magnetic field and to selectively perform a predefined function. The miniature device comprises a shell defining therewithin an internal cavity, and a magnetic arrangement disposed within the cavity. However, such devices do not form an elongated element having a proximal end portion of the steerable device opposed to a distal end portion remaining outside of the mammalian body for external manipulation.

US2019060608A1 discloses a catheter comprising an elongate shaft body and a tip member disposed at a distal end of the shaft body. The shaft body can include a liner, a braid member surrounding the liner, a multi-layer coil surrounding the braid member, and a polymer cover surrounding the multi-layer coil. An outer surface portion of the polymer cover can include one or more helical threads, which can be enveloped in an outer wrapper.

EP3854328A1 discloses a dilator that is capable of advancing and/or retreating by a rotation of a hollow shaft, and is also capable of smoothly performing a movement when being pushed in and/or pulled out linearly. The dilator includes a hollow shaft including a tapered section having a diameter increasing from a distal end toward a proximal end, and a spirally-arranged protruding portion provided on an outer peripheral surface of the hollow shaft and including gaps between neighboring sections thereof along an axial direction of the hollow shaft.

EP 3 772 317 A1 relates to an endoscope for inspection of the gastrointestinal tract of a mammal, in particular of the small intestine of a human, comprising an elongated, flexible tubular structure and a flexible, elongated element placed inside the tubular structure such that a distal portion of the elongated element protrudes from a distal end of the tubular structure and is connected to an endoscopic tip. The elongated element is engaged with the tubular structure in such way that a rotation of the tubular structure around the elongated element in a first rotation direction will increase a gap between the endoscopic tip and the distal end of the tubular structure, i.e. the endoscope becomes longer, while a rotation of the tubular structure around the elongated element in a second rotation direction, opposite to the first rotation direction, will decrease the gap between the endoscopic tip and the distal end of the tubular structure, i.e. the endoscope becomes shorter.

In one embodiment disclosed in EP 3 772 317 A1 , the tubular structure comprises an outer spiraling surface structure, i.e. a groove that spirals along the length of the tubular structure, which forms a continuous structure. During the manipulation of the endoscope, a physician transmits a rotation applied to the tubular structure to the elongated element to move forward the endoscopic tip. The movement of the endoscope is the result of successive elongation and contraction of the endoscope, which length is variable. The movement of the endoscopic tip is only indirectly connected to the rotation applied by the physician to the tubular structure. Indeed, a movement of the tubular structure can either correspond to a movement of the endoscopic tip away from the tubular structure remaining in place or to a movement of the tubular structure in the direction of the endoscopic tip remaining in place. In case miniaturization of the device is necessary, the construction itself of the endoscope limits the possibilities, in particular the reduction of its diameter because of the tubular structure surrounding the elongated that is required to transmit movement. It follows that this concept is not appropriate for interventional purposes in the vascular system requiring diameter typically of less than 4 mm, let alone in human brain requiring catheter even thinner than 2 mm.

It is an object of the present invention to provide a device for use inside of a mammalian body, which can be used in a reliable and reproducible manner in a wide variety of applications for inspecting and/or operating, diagnostic and/or interventional purposes inside of the mammalian body, and which is not subject to the limitations of the devices listed above, especially the tradeoff between flexibility and pushability.

This objective is met according to the present invention by providing a device with the features of claims 1 or 10. Further advantageous embodiments of the invention are the subject of the dependent claims.

The steerable device can be a needle, guidewire, catheter, endoscope, or the like that is used inside of a mammalian body, in particular of a human, for example to inspect and/or operate. The steerable device may be used for inspecting and/or operating inside of organs (e.g. liver, lungs, kidney, brain, etc.), inside of a body cavity (e.g. abdomen, spinal cord, sinuses etc.), or in the vasculature.

The device comprises an elongated element, which is elongated along a device longitudinal axis and configured to enter the soft tissue of the mammalian body through an incision or other suitable means. A device tip can be placed at a distal end of the elongated element. In particular, the elongated element ends distally in the device tip. The device tip may contain or carry measurement and/or actuating devices, such as a camera, sensors, ablation tip, needle, electrodes, etc. The device may contain an inner lumen that allows delivery of fluids, needles, biopsy tools, etc. to a target site. An end of the device opposed to the device tip, i.e. a proximal end of the device, is designed to remain outside of the mammalian body for external manipulation for example by a physician or a robot.

Further, the steerable device has a fixed length, i.e. the length of a given steerable device according to the invention remains the same during an intervention in contrast to the device disclosed in EP 3 772 317 A1 in which successive variations of the device length allow its progression.

The steerable device further comprises a magnetic element placed in a distal end portion of the elongated element and configured for allowing steering of the elongated element by way of an external magnetic field. A proximal end portion of the steerable device opposed to the distal end portion remains outside of the mammalian body for external manipulation of the steerable device. In a preferred embodiment, the magnetic element forms the foremost end of the steerable device to allow for an optimal steering of the steerable device. Preferably, the magnetic element is a permanent magnetic element. In a preferred embodiment, the magnetic element is arranged in the device tip comprising or being composed of, at least one magnetically responsive element so that an external magnetic field can control the orientation of the device tip. In a preferred embodiment, the magnetic element comprises a plurality of magnetic components that are distributed along the distal end portion of the elongated element to form a magnetic element having bending points between the magnetic components and being therefore flexible.

One important limiting factor that hinders traditional flexible medical devices from advancing in the soft tissue of a mammalian body is friction: after some distance, the friction between the elongated element and the soft tissue can become too high to continue effectively advancing the device. In this case, the elongated element either cannot be pushed any further or buckles, both of which prevent further advancement of the distal tip. All flexible medical devices that rely on traditional steering and pushing suffer from this tradeoff between flexibility and pushability.

According to an aspect of the invention, the steerable device comprises a structured section arranged proximal to the magnetic element, the structured section comprising a plurality of elongated protrusions formed on an outer surface of the elongated element and having each a protrusion longitudinal axis, said protrusion longitudinal axis forming an angle with the device longitudinal axis of the elongated element. The plurality of elongated protrusions forms a plurality of angles with the device longitudinal axis, wherein the plurality of angles are each less than 90°. The angles are measured in the clockwise direction from the device longitudinal axis oriented in the direction from proximal to distal. The arrangement is such that rotation of the elongated element in the clockwise direction around the device longitudinal axis effects a movement of advancement of the elongated element relative to the soft tissue along the device longitudinal axis.

The structured section has a greater surface rugosity than the surface of the rest of the elongated element that is unstructured.

The principle underlying the invention is to combine in the steerable device the structured section with the magnetic element. Rotation of the structured section as disclosed above produces advancement of the elongated element inside the soft tissue, and the magnetic element arranged distal to the structured section allows the steerable device to be steered precisely.

The magnetic element is typically less flexible than the elongated element. Further, the elongated element with a structured section is also less flexible than without a structured section. Therefore, it is advantageous to arrange the structured section proximal to the magnetic element to avoid an overlap of the structured section and the magnetic element. As a result, the flexibility of the steerable device is not unduly reduced and the progression for example in vessels or at branching vessels remains precise. More preferably, the magnetic element is consequently arranged in an unstructured section. As a result, the distal end portion can be steered by way of the magnetic field with a limited friction of the device.

In an embodiment in which the structured section is arranged proximal to the magnetic element, it is possible that a distal end portion of the structured section overlaps at least partially the magnetic element. As a result, the distal end portion can be slightly stiffened by the elongated protrusions and the magnetic element and buckling is reduced.

The configuration of the structured section supports the progression of the elongated element by way of the plurality of elongated elements when the elongated element is rotated in the clockwise direction. At the same time, the structured section remains flexible, in particular more flexible than a section of the same length having a continuous thread, and has a design that is simple to manufacture.

The rotary locomotion creates a pushing force all along the length of the elongated element where the structured section is engaged with surrounding soft tissue by way of the elongated element on the outer surface. The device can therefore be flexible and not suffer from the aforementioned problems normally associated with flexible medical devices. Magnetic steering allows the device to be controlled even as it is being rotated, something that would be impossible with traditional devices whose steering is based on puller wires or pre-curved distal sections.

Advantageously, the structured section comprising a plurality of elongated protrusions can be manufactured with large tolerances regarding the orientation of the elongated protrusions as long as the angle formed with the device longitudinal axis is less than 90°. It results that the structured section can be manufactured easily and at low cost.

The plurality of angles are measured in the clockwise direction from the device longitudinal axis oriented in the direction from proximal to distal. In other words, when the elongated element is oriented from proximal to distal, the device longitudinal axis is oriented in the direction from 6 o'clock, corresponding to proximal, to 12 o'clock, corresponding to distal, as seen on a clock, and the angles between the device longitudinal axis and the protrusion longitudinal axis are measured from 12 o'clock in the clockwise direction.

In a preferred embodiment, the plurality of angles have a single angle value chosen between 5° and 80°, i. e. all angles of the plurality of angles have the same value. The advantage of a single angle value is increased pushing forces along the device longitudinal axis, while the range disclosed allows the progression of the steerable device from proximal to distal when the steerable device is rotated in the clockwise direction. In a more preferred embodiment, the single angle value is between 10° and 60° leading to optimization of the pushing forces along the device longitudinal axis. In an even more preferred embodiment, the single angle value is more preferably between 20° and 30°.

This range has shown experimentally excellent advancement of the steerable device. It is noted that the term single angle value must be understood as a single value within the manufacturing tolerances. Therefore, it can include more than one value, wherein the values are distributed about central value, the single angle value, within the manufacturing tolerances.

The elongated protrusions can have the shape of an ellipse which major axis forms the protrusion longitudinal axis. Other elongated shapes are also possible, for example a rectangular shape which long side defines a protrusion longitudinal axis.

The plurality of elongated protrusions are separated from each other, i.e two elongated protrusions are free from a contact region common to each other.

Further, the plurality of elongated protrusions have each time a circumferential length shorter than the elongated element circumference in the structured region where the elongated protrusions are arranged. In other words, the elongated protrusions extent over less than one turn around the elongated element as seen in the longitudinal direction. They extend only over a portion of the elongated element circumference and are separated from each other. This is contrast to a spiraling structure having a continuous extension in the longitudinal direction over more than one turn.

The circumferential extension of the elongated protrusions can be characterized each time by a circular sector measured in a plane perpendicular to the device longitudinal axis and subtended by the projection in this plane of the radii crossing the proximal end and the distal end of the elongated protrusions, respectively. Because the plurality of elongated protrusions have a longitudinal length shorter than the elongated element circumference as mentioned above, the circular sector is less than 360°.

In a preferred embodiment, the circular sectors of the plurality of the elongated protrusions ranges from 30° to 150°. Preferably, the circular sector has an angle of 60° to 120°. On the one hand, the extension of the elongated protrusions is broad enough to engage with surrounding soft tissue, and on the other hand, the extension of the elongated protrusions is not broad enough to significantly reduce the flexibility of the steerable device in the structured section. More preferably, the circular sector has an angle of 100°. This configuration optimizes both aspects previously mentioned.

In a preferred embodiment, two consecutive elongated protrusions of the plurality of elongated protrusions each time overlap circumferentially at least partially. In other words, the circular sector angles subtended by two consecutive recesses overlap. Preferably, the circular sector angles overlap each time by 10% to 30% of the circular sector angles. More preferably, the circular sector angles overlap each time by 20%. The overlapping of two consecutive elongated protrusions forms a continuous succession of regions engaging with surrounding soft tissue. The result is the formation of a continuous engaging with surrounding soft tissue that promotes advancement of the steerable device. It must be noted that the overlap of the circular sector angles subtended by two consecutive elongated protrusions does not imply that the elongated protrusions have an intersection. Indeed, the provision of an intersection also depends on the axial extension of the elongated protrusions.

In a preferred embodiment, the plurality of elongated protrusions are aligned along a thread path having a thread path angle equal to the single angle value. The thread path angle is defined as the angle of the thread path viewed side on, relative to the device longitudinal axis. In this embodiment, the thread path forms a thread helix that is discontinuous. The alignment of the elongated protrusions along a thread path, in particular a helical path, further improves the advancement capabilities of the steerable device while it is rotated. It ensures a distribution over the whole structured section of uniform force distribution around the structured section that improves the control of the advancement.

Preferably, the thread path extends in at least one turn from a proximal end to a distal end of the structured region. This embodiment ensures stability over the length of the structured section. More preferably, the helical path extends in a plurality of turns from the proximal end to the distal end. Experimental results have shown an optimized stability over the length of the structured section in this case.

In a further preferred embodiment, the plurality of elongated protrusions can overlap end-to-end and form a continuous thread on the outer surface of the elongated element. The continuous thread formed by the elongated protrusions ensures a uniform force distribution around the structured section that improves the control of the advancement.

In a preferred embodiment, the plurality of angles has at least two angle values, the angle values being chosen between 5° and 80°. In a further preferred embodiment, the at least two angle values are chosen between 20° and 60° More preferably, the at least two angle values are chosen between 20° and 30°. The advantage of at least two angle values is that pushing forces along the device longitudinal axis can be optimized depending on the angle with which the steerable device is in contact with soft tissues. If the elongated elements having one of the at least two angle values do not support the progression of the steerable device because the contact with soft tissues is not appropriate at this one angle, it is possible that the elongated elements having another value of the at least two angle values can support the advancement of the steerable device. In an even more preferred embodiment, the plurality of angles has two angle values, the two angle values being chosen between 20° and 60°, i. e. all angles of the plurality of angles are chosen in a set of two angle values. More preferably, the two angle values are chosen between 20° and 30°. An embodiment having only two angle values is a good compromise between improved progression and manufacturing complexity. In a more preferred embodiment, the two angle values are separated by 30° to 60°. The two angle values are separated from each other to provide for optimized contact possibilities with soft tissues.

In a preferred embodiment in which the plurality of angles has at least two angle values, the plurality of elongated protrusions are aligned along at least two thread paths having each time a thread path angle equal to an angle value of the at least two angle values. Preferably, the at least two thread paths extend in at least one turn from a proximal end to a distal end of the structured region. Features related to elongated protrusions discussed in relation to embodiments having one thread path above can apply equally to embodiments having at least two thread paths.

In a preferred embodiment, an outer tubular structure surrounds a section of the elongated element, the tubular structure comprising an engaging element configured to engage with the plurality of elongated protrusions such that rotation of the elongated element around its device longitudinal axis effects a movement of the elongated element relative to the tubular structure along the device longitudinal axis. The tubular structure will be described in more details in relation to a further embodiment below. The same effects and advantages related to the tubular structure apply for the present embodiment.

In a preferred embodiment, the plurality of elongated protrusions has a triangular cross section to provide for an improved contact with soft tissues. In a preferred embodiment, the plurality of elongated protrusions has a circular cross section to provide for a smooth contact surface with soft tissues. In a preferred embodiment, the plurality of elongated protrusions has a hump-shaped cross section, preferably approximatively in the form of a sinus curve, to provide for a smoother contact surface with soft tissues.

In a preferred embodiment, the structured section is interrupted by a plurality of unstructured sections that are free from elongated protrusions.

Unstructured sections have a higher flexibility while the elongated elements reduce flexibility in the structured sections. This embodiment allows to configure the steerable device with sections having higher flexibility, namely the unstructured sections, mitigating the reduction of flexibility of structured sections and consequently optimizing the advancement capacity of the steerable device.

In a more preferred embodiment, the unstructured sections of the plurality of unstructured sections have the same length and are separated from each other by a constant pitch. This simple design provides also for a homogeneous flexibility profile along the device longitudinal axis so that the steerable device can be easily controlled.

The elongated protrusions can be made of a material suitable for biomedical application. In particular, it can be a magnetically non-responsive material, for example a non-ferromagnetic material, to avoid interactions with the magnetic field applied to steer the elongated element. For example, suitable material can be a polymer material like nylon, polyurethane, polyethylene, polyether block amide (for example known under the tradename Pebax®), or silicone.

Further, it is also disclosed an embodiment of the steerable device in which the flexible elongated element is elongated along a device longitudinal axis and configured to enter soft tissue of the mammalian body.

In addition, the magnetic element is placed in the distal end portion of the elongated element and configured for allowing steering of the elongated element by way of an external magnetic field. A proximal end portion of the steerable device opposed to the distal end portion remains outside of the mammalian body for external manipulation of the steerable device. The magnetic element being less flexible than the elongated element, it is advantageous to arrange it in the distal end portion that can be advanced more easily and oriented more precisely for example inside vessels or at branching vessels while steering the steerable device. The magnetic element comprises or is composed of, at least one magnetically responsive element so that an external magnetic field can control the orientation of the device tip. Preferably, the magnetic element is a permanent magnetic element.

The magnetic element reacts to an external magnetic field and thus allows controlled steering of the elongated element. The magnetic field can be produced by an apparatus placed outside of the mammal body or by any other suitable means.

In a further aspect of the invention, the steerable device comprises a structured section in the form of an outer thread structure, formed on the outer surface of the elongated element and having a radial extension beyond the outer surface of the elongated element. In a preferred embodiment, the thread structure is a helical thread structure. The thread structure can have the form of a spiral auger.

Further, the steerable device has a fixed length, i.e. the length of a given steerable device according to the invention remains the same during an intervention in contrast to the device disclosed in EP 3 772 317 A1 in which successive variations of the device length allow its progression. The diameter of the elongated element is essentially the same, preferably the same over the length of the steerable device.

According to the invention the thread structure is made of a magnetically non-responsive material and is configured to engage with soft tissue surrounding the elongated element inside the mammalian body such that rotation of the elongated element around its device longitudinal axis effects a movement of the elongated element relative to the soft tissue along the device longitudinal axis.

Rotation of the thread structure produces advancement of the elongated element inside the soft tissue, and the magnetic element allows the device to be steered. The configuration of the structured section, i.e. the thread structure, supports the progression of the elongated element when the elongated element is rotated in the clockwise direction. The rotary locomotion creates a pushing force all along the length of the elongated element where the thread structure is engaged with surrounding soft tissue by way of the elongated element on the outer surface. The device can therefore be extremely flexible and not suffer from the aforementioned problems normally associated with flexible medical devices. Magnetic steering allows the device to be controlled even as it is being rotated, something that would be impossible with traditional devices whose steering is based on puller wires or pre-curved distal sections.

The thread structure can be formed of a wire wound around the elongated element along its length. Alternatively, the thread structure may be extrusion formed at the outer wall of the elongated element.

The thread structure is made of magnetically non-responsive material to avoid interaction of the thread structure with the magnetic field applied to steer the magnetic element and that would cause deviations in the path of the steerable device. This embodiment allows an optimized steering. For example, a magnetically non-responsive material can be a non-ferromagnetic material.

However, it can also be a material having diamagnetic or paramagnetic properties which magnetic contribution is negligible compared to the magnetic contribution of the magnetic element to the steering of the device. These properties are such that the interaction of the outer thread device structure with the magnetic field is orders of magnitudes smaller than the interaction of the magnetic element with the magnetic field and, consequently, must not be considered while steering the steerable device in the mammalian body.

In a preferred embodiment, the thread structure has a triangular cross section to provide for an improved contact with soft tissues. In a preferred embodiment, the thread structure has a circular cross section to provide for a smooth contact surface with soft tissues. In a further preferred embodiment, the thread structure has a hump-shaped cross section, preferably approximatively in the form of a sinus curve, to provide for a smoother contact surface with soft tissues. The hump-shaped cross section has further the advantage to avoid the formation of cavities between the thread structure and the outer surface of the elongated element.

In a preferred embodiment, the thread structure is interrupted by a plurality of unstructured sections that are free from thread structure. Unstructured sections have a higher flexibility while the thread structure has a reduced flexibility. This embodiment allows to configure the steerable device with sections having a higher flexibility, namely the unstructured sections, mitigating the reduction of flexibility of the thread structure and consequently an optimized advancement of the steerable device.

Further, according to the invention, the thread structure is arranged proximal to the magnetic element. The magnetic element is typically less flexible than the elongated element. Further, the elongated element with a thread structure is also less flexible than without thread structure. Therefore, it is advantageous to avoid an overlap of the thread structure and the magnetic element such that the flexibility of the steerable device is not unduly reduced and the progression for example in vessels or at branching vessels remains precise. More preferably, the magnetic element is consequently arranged in an unstructured section. As a result, the distal end portion can be steered by way of the magnetic field with a limited friction of the device.

In a preferred embodiment, the radial extension of the thread structure measured from the outer surface of the elongated element has a first value in a first portion of the structured section and a second value different from the first value in a second portion of the structured section arranged proximal to the first portion such that the first portion and the second portion have a different flexibility. This embodiment allows to configure the flexibility of the thread structure itself. In a further preferred embodiment, the second value is larger than the first value and the radial extension is fading distally, towards the device tip, from the second value into the first value.

The thread structure extends radially beyond the outer surface of the elongated element. This has the advantage that the thread structure can embed itself into the soft tissue surrounding the elongated element and generate a locomotive force along the device longitudinal axis upon rotation of the elongated element around or about the device longitudinal axis.

Because the force pushing the elongated element forward is generated by its rotational engagement with soft tissue, the locomotive force is generated at all points along the elongated element where the thread structure is engaged with the soft tissue. The elongated element can therefore be designed with a higher flexibility and/or elasticity compared to devices that rely solely on proximal pushing for locomotion without the risk of the elongated element buckling. Furthermore, both a forward movement, towards a distal end of the device, and a backward movement, or retraction, towards a proximal end of the steerable device, can be much more exactly controlled because the angle of rotation determines very reliably the amount of linear movement.

The device longitudinal axis, or centerline, must then be interpreted as a line passing through the center of the elongated element, or in other words a line connecting the centers of all the cross-sectional circles that one obtains when viewing every cross-section of the elongated element along its length.

While the elongated element moves inside soft tissue, such as the brain or liver, it may be steered along or follow a curving path. In a preferred embodiment, the magnetic element is arranged in the device tip. Utilizing magnetic steering as opposed to steering based on the traditional puller wires allows the device to be much more flexible and allows it to be steered during rotation, which would be impossible with puller wires. In a preferred embodiment, the device tip forms the foremost end of the steerable device to allow for an optimal steering of the steerable device. In particular, the device tip may itself be magnetic and act as the magnetic element described herein. Because the device uses rotary locomotion to generate forward and backward movement, it can be more flexible than traditional devices that rely on proximal pushing/pulling forces for locomotion. This flexibility is critical for being able to effectively steer the device magnetically as the forces generated by magnetic steering are considerably lower than those involved in traditional steering methods (e.g. puller wires). Furthermore, when the device has been steered along a tortuous path, the nature of the rotary locomotion mechanism enables it to continue advancing or retracting without the risk of buckling or cutting the tissue. Together, the rotary locomotion mechanism and remote magnetic steering make the device capable of moving along complex and tortuous paths that would be impossible with traditional devices.

According to an advantageous embodiment, the thread structure is formed along at least 10%, 50%, or 80% of the length of the elongated element. The longer the thread structure is, the more the force advancing the elongated element will be distributed over the length of the elongated element.

The engagement of the thread structure with the soft tissue will stabilize the device, preventing it from being pushed backward if a tool is inserted through the device and pushed into a target site (e.g. for injection); and also allow a tool passed through the device to apply larger pulling forces to tissue at a target site (e.g. for taking a biopsy) than would otherwise be possible with such a flexible device. The elongated element may in some embodiments comprise two or more thread structures spaced apart along the length of the elongated element.

Preferably, the thread structure has a thread path angle of between 5° and 60°. In particular, the thread path angle of the thread structure should be such that the intermediate gaps between threads are sufficient to engage with the soft tissue.

Preferably, the overall diameter of the device is up to 15 mm for body cavities, up to 6 mm for organs, and up to 3 mm for the vasculature.

Advantageously, the thread structure has a thickness measured radially from the outer surface of the elongated element of between 5% to 15% of a diameter of the elongated element, in particular of an outside diameter of the elongated element.

In a preferred embodiment, the magnetic element comprises a plurality of magnetic components that are distributed along the distal end portion of the elongated element to form a magnetic element having bending points between the magnetic components and being therefore flexible.

In a preferred embodiment, a section of the elongated element is surrounded by an outer tubular structure, such as a sheath. The tubular structure may be flexible. The elongated element and the tubular structure may in particular be arranged coaxially or substantially coaxially.

Advantageously, the tubular structure comprises an engaging element configured to engage with the thread structure such that rotation of the elongated element around its device longitudinal axis effects a movement of the elongated element relative to the tubular structure along the device longitudinal axis. In particular, the engaging element, such as a protruding element like a pin, peg, or bearing ball, may reach into the threads of the thread structure or, if applicable, into the plurality of elongated protrusions, to translate a rotation of the elongated element with respect to the engaging element into a translational movement of the elongated element with respect to the engaging element. In particular, the engaging element may be a female thread, having only a few windings, that is complementary to the thread structure and disposed at the inner wall of the tubular structure. This feature may aid in the controlled advancement of the steerable device because the tubular structure may act as a temporary anchor against which the elongated element can push forward or backward. The tubular structure may add rigidity to the device and/or reduce amount of thread or, if applicable, the plurality of elongated protrusions, in contact with the soft tissue.

In a preferred embodiment, the engagement element may be designed to seal against the thread structure, preventing backflow of bodily fluids, or may be designed to freely allow fluids pumped into the tubular structure to pass into the body.

Advantageously, the elongated element passes freely through an inner lumen of the tubular structure and is attached to the tubular structure only by means of this rotational engagement mechanism. In particular, the elongated element is not limited in its range of linear motion by its attachment to the tubular structure. Preferably, a section of the elongated element extending out of the tubular structure and disposed between the device tip and a distal end of the tubular structure is exposed, in particular exposed to the environment of the surrounding soft tissue, i.e. without being protected by other parts of the steerable device.

In a preferred embodiment, the thread structure is freely wound around the elongated element, wherein a distal end of the thread structure is fixed to the elongated element and a proximal end of the thread structure is removably fixed to the elongated element to provide for a different thread pitch by displacing proximally or distally the proximal end in the longitudinal direction, i.e. by extension or compression of the thread structure. This embodiment has the advantage that when the contact with soft tissues is not appropriate to support progression at a given thread pitch, it is possible to modify, i.e. either compress or extend or both for example in alternating sequences, the thread pitch to better support the advancement of the steerable device.

In a preferred embodiment, the thread structure is freely wound around the elongated element and is secured to the elongated element by locking means that engages the thread structure in a first direction of rotation of the elongated element to transfer rotational movement of the elongated element to the thread structure and that disengages the thread structure in a second direction of rotation opposed to the first direction of rotation to allow free rotation of the thread structure. Advantageously, this embodiment allows controlling of friction with surrounding soft tissue that are detrimental to a safe advancement of the steerable device.

In a further preferred embodiment, the locking means is retractable to allow a free rotation of the thread structure in the first direction of rotation and in the second direction of rotation. Advantageously, this embodiment allows a release of the outer thread engaging with surrounding soft tissue in case it is blocked or causing too much friction with surrounding soft tissue that are detrimental to a safe advancement of the steerable device.

The elongated element should have a flexibility that is sufficient for it to follow bending paths along the soft tissue in the body. On the other hand, it should have enough rotational rigidity to allow torque that is being applied on a proximal section of the elongated element to be transmitted directly along the length of the elongated element to a more distal section, where the thread structure is located. Such direct torque transmission is especially important for the rotary locomotion mechanism described above. Longitudinal flexibility on the one hand and torsional rigidity on the other hand may be achieved by a combination of material and design properties of the elongated element, such as the use of multiple tubular layers and hollow stranded-wire coils as described in the following.

Advantageously, the elongated element comprises multiple tubular layers that are arranged coaxially. Advantageously, at least 2 or 3 coaxially arranged tubular layers are stacked radially, i.e. along the thickness of the elongated element.

In a preferred embodiment, the elongated element comprises at least one tubular layer formed of multiple wires, in particular metal wires, e.g. made of a stainless steel alloy, wound in a helical fashion in the direction of the device longitudinal axis along a tubular surface, such that the multiple wires are tightly packed in a singular row. In other words, in a cross-sectional view of the tubular layer, where the cross-sectional plane is perpendicular to the device longitudinal axis of the elongated element, each wire contributes a wire cross-section to the circumference of the tubular layer. Preferably, the tubular layer is formed of at least 7, 9 or 11 wires. Such a tubular layer may be called a wire-stranded hollow coil body as described in EP 1 428 547 A2 or as a hollow stranded-wire coil as described in EP 2 263 736A1 .

In advantageous embodiments, the wires of one of the tubular layers are wound in a direction opposite to the wires of another of the tubular layers.

This means that when looking at the cross-section of the elongated element and following it along the length of the elongated element, a wire wound in one tubular layer rotates in one direction and a wire wound in the other tubular layer rotates in the opposite direction. Preferably, the tubular layers with wires wound in opposite directions are arranged immediately adjacent to each other in the elongated element. Further preferred, the winding direction of consecutive tubular layers alternate from one tubular layer to a neighboring one across three or more tubular layers.

According to another embodiment, the elongated element comprises a tubular layer with perforations along its length and circumference. The size and distribution of the perforations on the tubular layer can determine the flexibility of the elongated element. Alternatively or in addition to the perforations, the tubular layer may comprise indentations that are also distributed along the length and circumference of the tubular layer. In particular, the distribution of the perforations or indentations along the length of the tubular layer may be cyclical.

Advantageously, the tubular layer comprises slits that extend along its surface in an annular direction or in a helical direction. Each slit may extend over the entire circular circumference of the tubular layer, interrupted at two or more points by bridges.

The elongated element can comprise multiple sections along its length that have different flexibilities. For example, a distal section, towards the device tip, may be of increased flexibility compared to the rest of the elongated element to allow for more effective magnetic steering in tight.

The elongated element may comprise an inner lumen, through which medical instruments-such as needles, biopsy tools, etc.—and fluids—such as drugs, contrast agent, coagulants, solutions, etc.—may be delivered at the desired site in the mammalian body, in particular through the lumen's distal termination at the device tip.

Further, it is also disclosed the use of a steerable device according to any one of the previous embodiments disclosed above with a medical system comprising a magnetic field generator designed to generate a magnetic field of force with a predetermined direction, amplitude and spatial variation for application to the steerable device and a unit designed to apply a rotational movement to the steerable device. The medical system allows a wide variety of applications for inspecting and/or operating, diagnostic and/or interventional purposes inside of the mammalian body. Preferably, the magnetic field of force generator is in the form of a magnetic navigation system with multiple electromagnets or permanent magnets located and movable around the patient providing an inhomogeneous magnetic field at predetermined positions in the space, in which the steerable device is moved.

Herein, the expressions proximal and distal are used with respect to the position of the physician utilizing the steerable device. The expression distal thus describes parts, sections or directions that are oriented or placed towards the inside of the patient's body, where the physician cannot manipulate the parts directly with his hands. Likewise, the expression proximal stands for the parts, sections or directions positioned at or oriented towards the location of the physician. In particular, the proximal end refers to the end of the elongated element that in use will be extending out of the incision, i.e. a proximal end of the elongated element extends out of the mammalian body in use, while the distal end of the elongated element refers to the end of the elongated element inside the patient during a procedure.

### DESCRIPTION OF FIGURES

Some examples of embodiments of the present invention will be explained in more detail in the following description with reference to the accompanying schematic drawings, wherein:

FIG. 1 shows a cross-sectional view of a steerable device according to a first embodiment;

FIG. 2 shows a cross-sectional view of a steerable device according to a second embodiment;

FIG. 3 shows a cross-sectional view of a steerable device according to a third embodiment;

FIG. 4 shows a cross-sectional view of a steerable device according to a fourth embodiment;

FIG. 5 shows a cut-away view of an elongated element covered by a thread structure according to a fifth embodiment;

FIG. 6 shows a tubular layer of an elongated element according to a sixth embodiment;

FIG. 7 shows a perspective view of a steerable device according to a seventh embodiment with a structured section;

FIG. 8 shows a detailed side view of a structured section according to a preferred embodiment;

FIG. 9 shows a detailed side view of a structured section according to a further preferred embodiment;

FIG. 10 shows a detailed side view of a structured section according to a further preferred embodiment;

FIG. 11 shows a side view of a thread structure of the steerable device according to a preferred embodiment;

FIG. 12 shows a side view of a thread structure of the steerable device according to a further preferred embodiment;

FIG. 13 shows a side view of a thread structure of the steerable device according to a further preferred embodiment;

FIG. 14 shows a cross section of a thread structure of the steerable device according to a preferred embodiment;

FIG. 15 shows a cross section of a thread structure of the steerable device according to a further preferred embodiment; and

FIG. 16 shows a cross section of a thread structure of the steerable device according to a further preferred embodiment.

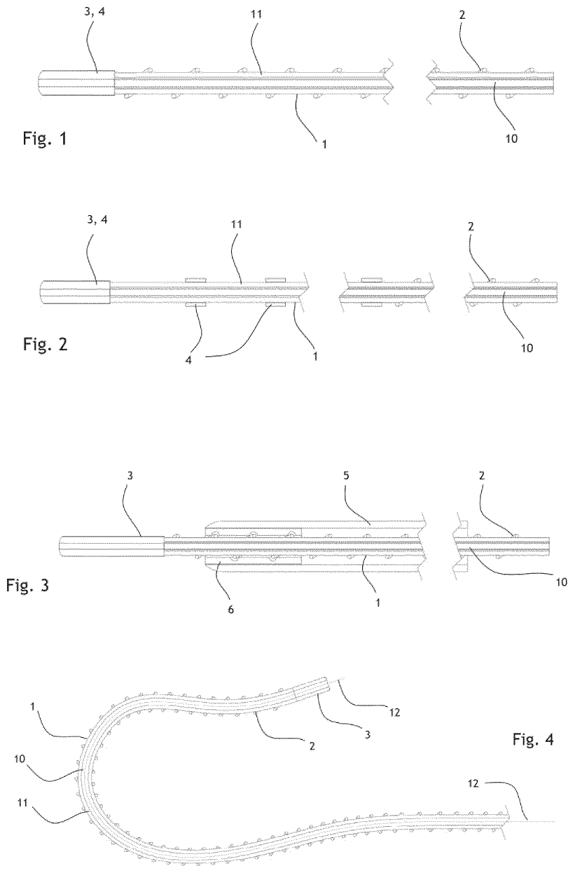

FIG. 1 shows a cross-sectional view of a steerable device according to one possible embodiment. In this embodiment, the device comprises an elongated element 1, which has a device tip 3 at its distal end. The elongated element 1 has a wall 11 that encloses an inner lumen 10, which extends along a device longitudinal axis. Various medical instruments and/or substances can be fed through the inner lumen 10 of the elongated element 1 and exit from the device tip 3 to a desired site in the body.

The elongated element 1 has a structured region in the form of a thread structure 2 surrounding the outer surface of the elongated element 1. The thread structure 2 is formed as a spiral auger, which is helically wound on the outer surface of the elongated element 1 and extends up to the device tip 3.

When the device is inserted into soft tissue of a mammalian body through an incision, e.g. into an organ, body cavity, blood vessel, or other soft tissue, the thread structure 2 engages with the tissue surrounding the elongated element 1 such that a rotation of the elongated element 1 will lead to an advancement or retraction of the elongated element along its device longitudinal axis. The device may advance in a forward or a backward direction depending on the direction of rotation of the elongated element 1.

The device tip 3 is magnetic, i.e. it is made partly or completely of a magnetically responsive material. This is one possible implementation of having a magnetic element 4 placed at or near the device tip 3.

A slightly different embodiment of the steerable device is shown in FIG. 2. Here, the thread structure 2 does not extend all the way up to the device tip 3. Instead, magnetic element 4 designed as a plurality of magnetic components in the form of magnet rings are placed, here equidistantly, along the gap between the device tip 3 and the distal end of the thread structure 2. The magnet rings will increase the effect of a magnetic field on the steering of the device while keeping flexibility of the elongated element between the magnet rings.

A further embodiment of the steerable device having a similar construction to the embodiment of FIG. 1 is disclosed in FIG. 11, in which the thread structure 2 is interrupted by a plurality of unstructured sections 106 that are free from the thread structure and have therefore a higher flexibility.

In the embodiment of the steerable device having a similar construction to the embodiment of FIG. 1 and disclosed in FIG. 12, the radial extension of the thread structure 2 measured from the outer surface of the elongated element 1 has a first value in a distal portion 108 of the structured section and a second value larger than the first value in a proximal portion 110 of the structured section, the radial extension fading distally, towards the tip of the steerable device, from the second value into the first value. In an embodiment in which the cross section of the thread structure 2 is circular, the radial extension corresponds to the diameter of the thread structure 2 that has a larger value in the distal portion 108 than in the proximal portion 110.

In the embodiment of the steerable device having a similar construction to the embodiment of FIG. 1 and illustrated in FIG. 13, the thread structure 2 has a thread pitch 112 which value increases from distal to proximal resulting in a flexibility increasing from distal to proximal.

Yet another embodiment of the steerable device is shown in FIG. 3. This embodiment distinguishes itself from the one shown in FIG. 1 in that a section of the elongated element 1 is surrounded by an outer tubular structure 5. The tubular structure 5 comprises an engagement element 6 that engages a section of the thread structure 2 such that relative rotation between the elongated element 1 and the tubular structure 5 results in a translational motion of the elongated element 1 relative to the tubular structure 5. This feature may aid in anchoring the device and improving the ability of the elongated element to be advanced and/or retracted, as well as provide another lumen through which to pump fluids.

FIG. 4 shows a cross-sectional view of an embodiment of the steerable device, such as the embodiment of FIG. 1, in a curved state or bent state. As can be seen here, the device longitudinal axis 12 of the elongated element 1 can be a curved line if the elongated element 1 is not in a straightened state.

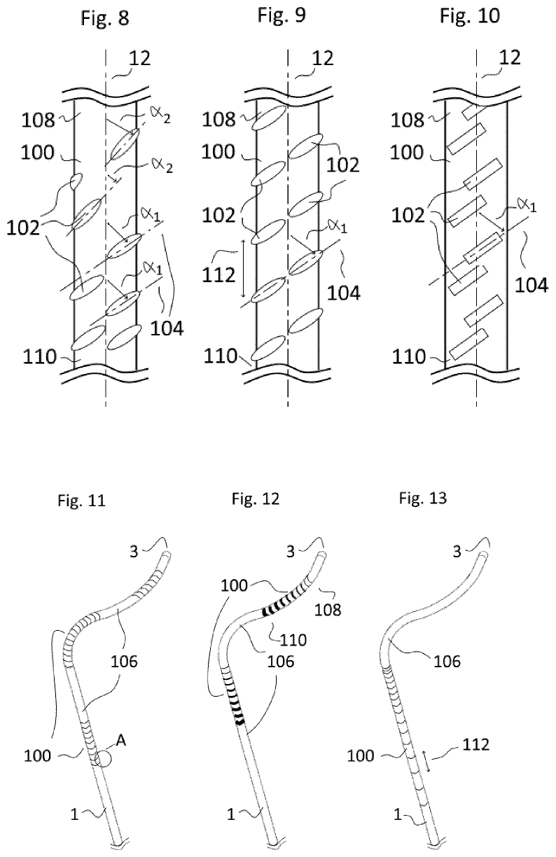

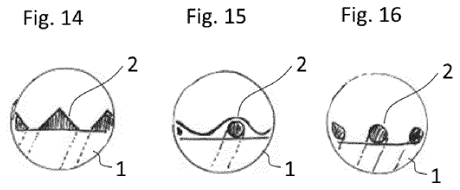

Cross sectional views of possible and not limiting embodiments of the thread structure are disclosed in FIG. 14, FIG. 15 and FIG. 16 and referring to the zoomed area A of the exemplary embodiment of the steerable device represented in FIG. 11. It is noted that the cross sections disclosed are also possible cross sections for elongated protrusions similar to those disclosed in FIG. 8, FIG. 9 and FIG. 10. In FIG. 14, the thread structure 2 has a triangular cross section to provide for an improved contact with soft tissues. In FIG. 16, the thread structure 2 has a circular cross section to provide for a smooth contact surface with soft tissues. In FIG. 15, the thread structure 2 has a hump-shaped cross section in the form of a sinus curve to provide for a smoother contact surface with soft tissues and avoid the formation of cavities between the thread structure 2 and the outer surface of the elongated element.

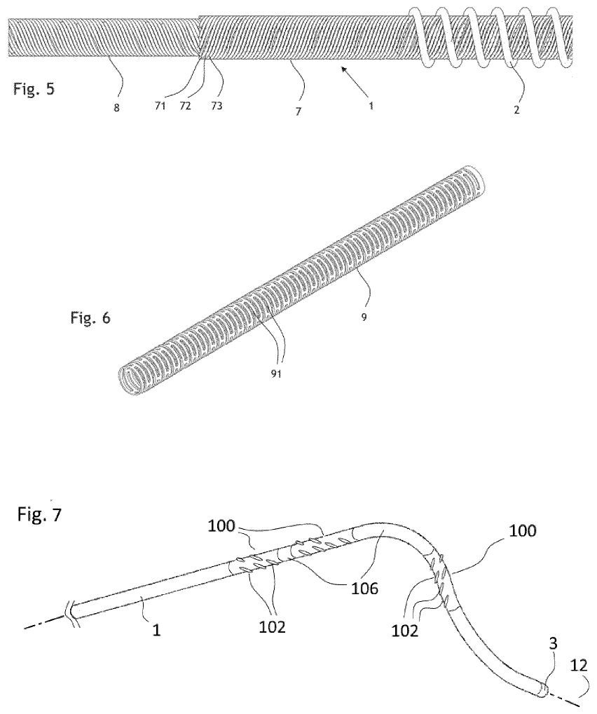

A detailed view of the structure of the elongated element 1 according to a preferred embodiment is shown in FIG. 5. The elongated element is formed of a first tubular layer 7 and a second tubular layer 8, which has a smaller diameter and is fitted within the first tubular layer 7. FIG. 5 shows a cut-away view, in which a left part of the first tubular layer 7 is removed to show the second tubular layer 8 hidden underneath.

Each tubular layer 7, 8 is formed of multiple wires. As an example, the first tubular layer 7 is formed of twelve individual wires 71, 72, 73 each of which is helically wound to form the surface of the tubular layer 7. Together, the wires 71, 72, 73 form the first tubular layer 7. The second tubular layer 8 is formed in a similar fashion, but with a smaller overall diameter. Furthermore, the wires of the first tubular layer 7 are wound in the opposite direction as the wires of the second tubular layer 8. The helical structure 2 is formed on the outer surface of the first tubular layer 7. It consists of a single wire that is wound in a similar fashion as any of the individual wires 71, 72, 73 of the first tubular layer 7.

In FIG. 6 a tubular layer 9 according to a different embodiment for use in an elongated element 1 is shown. This tubular layer 7 is made of a solid tube which is perforated to control the bending stiffness of the tubular layer. The perforations 91 are formed as annular slits along the length of the tubular layer 9. Multiple coaxially-arranged tubular layers of this kind may be combined to form the elongated element 1. Alternatively, tubular layers of this kind may be combined with one or more tubular layers 7, 8 shown in FIG. 5.

The steerable device disclosed in FIG. 7 comprises an elongated element 1 elongated along a device longitudinal axis 12 and a device tip 3 arranged at a distal end of the elongated element 1. The device tip 3 is magnetic, i.e. it is made partly or completely of a magnetically responsive material, to allow steering of the steerable device in a magnetic field.

Further, the steerable device comprises a structured section 100 comprising a plurality of elongated protrusions 102. In the present embodiment, the structured section 100 is interrupted by a plurality of unstructured sections 106, here two unstructured sections, that are free from elongated protrusions. As can be seen in more details in FIG. 8, FIG. 9 and FIG. 10 representing each time a portion of possible embodiments of the structured section 100, each elongated protrusion 102 of the plurality of elongated protrusions 102 defines a protrusion longitudinal axis 104. The portion has a proximal end 110 and a distal end 108. The protrusion longitudinal axis 104 defines each time an angle α1 (alpha 1), α2 (alpha 2) with the device longitudinal axis 12, the plurality of elongated protrusions 102 forming a plurality of angles, wherein the plurality of angles are each less than 90°, as measured in the clockwise direction from the device longitudinal axis 12 oriented in the direction from proximal to distal. In other words, in FIG. 8, FIG. 9 and FIG. 10, the direction from proximal to distal corresponds to the direction from the bottom to the top of the figure. As a result of this arrangement of the elongated protrusions, a rotation of the elongated element 1 around the device longitudinal axis 12 effects a movement of the elongated element relative to the soft tissue along the device longitudinal axis. For the sake of clarity, only a limited number of protrusion longitudinal axis 104 and angles α1, α2 are represented.

In the embodiment of FIG. 8 and FIG. 9, the elongated protrusions have essentially the form of an ellipse, wherein the ellipse major axis defines the protrusion longitudinal axis 104. In the embodiment of FIG. 8, the plurality of angles has two angle values α1, α2, wherein α1 is about 60° and α2, is about 30°. In this embodiment, the angle α1 and the angle α2 have a value difference of about 30°from each other. In the embodiment of FIG. 9, the plurality of angles has a single angle value α1. Further, the plurality of elongated protrusions 102 are aligned along a thread path having a thread path angle equal to the angle value α1 and a thread pitch 112. In this embodiment, the thread path forms a discontinuous thread helix, of which only the visible side is represented.

In the embodiment of FIG. 10, the elongated protrusions have essentially the form of a rectangle, wherein the length of the rectangle defines the protrusion longitudinal axis 104 forming an angle α1 with the device longitudinal axis 12. It is noted that the plurality of angles has the single angle value α1.

### REFERENCE NUMERALS

1 elongated element
10 inner lumen of elongated element
11 wall of elongated element
12 device longitudinal axis
2 outer thread structure
3 device tip
4 magnet rings
5 tubular structure
6 engagement element
7 (first) tubular layer
71, 72, 73 wires
8 second tubular layer
9 tubular layer, further embodiment
91 annular slit, perforation
100 structured section
102 elongated protrusions
104 protrusion longitudinal axis
106 unstructured section
110 proximal end of structured section
108 distal end of structured section
112 thread pitch

1. A steerable device for use inside of a mammalian body, comprising:
a flexible elongated element, elongated along a device longitudinal axis and configured to enter soft tissue of the mammalian body;
a magnetic element placed in a distal end portion of the elongated element and configured for allowing steering of the elongated element by way of an external magnetic field, a proximal end of the elongated element extending out of the mammalian body in use,
wherein a structured section arranged proximal to the magnetic element and comprising a plurality of elongated protrusions formed on an outer surface of the elongated element and having each a protrusion longitudinal axis, each protrusion longitudinal axis forming an angle with the device longitudinal axis, the plurality of elongated protrusions forming a plurality of angles with the device longitudinal axis, wherein each angle of the plurality of angles is less than 90°, as measured in the clockwise direction from the device longitudinal axis oriented in the direction from proximal to distal, such that rotation of the elongated element in the clockwise direction around the device longitudinal axis effects a movement of advancement of the elongated element relative to the soft tissue along the device longitudinal axis

2. The device according to claim 1, wherein the plurality of elongated protrusions extent each over less than one turn around the elongated element as seen in the longitudinal direction.

3. The device according to claim 1, wherein the plurality of angles have each a same single angle value chosen between 5° and 80°.

4. The device according to claim 3, wherein the plurality of elongated protrusions are aligned along a thread extending along the elongated element, the thread having a thread path angle equal to the single angle value.

5. The device according to claim 1, wherein the plurality of angles are distributed according to at least two angle values, the at least two angle values being chosen between 5° and 80°.

6. The device according to claim 5, wherein the plurality of elongated protrusions are aligned along at least two threads having each time a thread path angle equal to an angle value of the at least two angle values.

7. The device according to claim 3, wherein an outer tubular structure surrounds a section of the elongated element, the tubular structure comprising an engaging element configured to engage with the plurality of elongated protrusions such that rotation of the elongated element around its device longitudinal axis effects a movement of the elongated element relative to the tubular structure along the device longitudinal axis.

8. The device according to claim 1, wherein the plurality of elongated protrusions have a triangular cross section or a circular cross section or a hump-shaped cross section.

9. The device according to claim 1, wherein the structured section is interrupted by a plurality of unstructured sections that are free from elongated protrusions.

10. The steerable device for use inside of a mammalian body, comprising:
a flexible elongated element, elongated along a device longitudinal axis and configured to enter soft tissue of the mammalian body;
a magnetic element placed in a distal end portion of the elongated element and configured for allowing steering of the elongated element by way of an external magnetic field, a proximal end of the elongated element extending out of the mammalian body in use,
a structured section in the form of an outer thread structure being formed on an outer surface of the elongated element and having a radial extension beyond the outer surface of the elongated element, wherein the thread structure is arranged proximal to the magnetic element and made of a magnetically non-responsive material, wherein the thread structure is configured to engage with soft tissue surrounding the elongated element inside the mammalian body such that rotation of the elongated element around its device longitudinal axis effects a movement of the elongated element relative to the soft tissue along the device longitudinal axis.

11. The device according to claim 10, wherein the thread structure has a triangular cross section or a circular cross section or a hump-shaped cross section.

12. The device according to claim 10, wherein the structured section is interrupted by a plurality of unstructured sections that are free from the thread structure.

13. The device according to claim 10, wherein the radial extension of the thread structure measured from the outer surface of the elongated element has a first value in a first portion of the structured section and a second value different from the first value in a second portion of the structured section such that the first portion has a different flexibility from the second portion.

14. The device according to claim 10, wherein a the thread structure is freely wound around the elongated element, wherein a distal end of the thread structure is fixed to the elongated element and a proximal end of the thread structure is removably fixed to the elongated element to provide for a different thread pitch by displacing proximally or distally the proximal end in the longitudinal direction.

15. The device according to claim 10, wherein the thread structure is freely wound around the elongated element, wherein the elongated element comprises locking means that engages the thread structure in a first direction of rotation of the elongated element to transfer rotational movement of the elongated element to the thread structure and that disengages from the thread structure in a second direction of rotation opposed to the first direction of rotation to allow free rotation of the thread structure.
